(19) United States
(12) Patent Application Publication
(10) Pub. No.: US 2018/0179129 A1
(43) Pub. Date: Jun. 28, 2018

(54) SOLVENT COMPOSITIONS FOR USE AS HEXANE REPLACEMENTS

(71) Applicant: TBF ENVIRONMENTAL TECHNOLOGY INC., Surrey (CA)

(72) Inventors: David Anthony Pasin, Vancouver (CA); Shira Devorah Bogner, Vancouver (CA)

(21) Appl. No.: 15/577,126

(22) PCT Filed: May 26, 2016
(86) PCT No.: PCT/IB2016/053076

§ 371 Date: Nov. 27, 2017

Related U.S. Application Data
(60) Provisional application No. 62/168,508, filed on May 29, 2015.

(51) Int. Cl. C07C 25/13; C07C 69/003; C07F 7/08

(52) U.S. Cl. CPC C07C 25/13; C07F 7/0849; C07C 69/003

(57) ABSTRACT

The present disclosure provides, in part, a solvent composition for use as a hexane replacement. The solvent composition may include para-Chlorobenzotrifluoride (PCBTF), a methylated organosilicon compound, and an acetate ester.

### FIELD OF INVENTION

The present disclosure relates generally to solvent compositions. More specifically, the present disclosure relates to solvent compositions that may be used to replace hexane.

### BACKGROUND OF THE INVENTION

Organic solvents, such as hexane, heptane, methyl ethyl ketone (MEK), acetone, xylene and toluene, and other hydrocarbons or oxygenated solvents are used in a number of applications. Many of these solvents have toxic and/or environmentally deleterious properties. For example, human and animal studies indicate that exposure to these chemicals can have detrimental effects on the central nervous system, as well as on the hepatic and renal systems. Hexane, xylene, toluene and perchloroethylene are used as a special-use solvent and as a cleaning agent. For example, hexane, xylene, toluene and perchloroethylene are used in the formulation of glues for shoes, leather products, and roofing materials, and in textile and water-proofing compound manufacturing. Commercial grades of hexane, xylene, toluene and perchloroethylene are used as solvents for rubber cement, adhesives, varnishes, and inks. Hexane, xylene, toluene and perchloroethylene are also used as cleaning agents (degreasers) in the automotive, brakes and printing industries.

“Hazardous air pollutants” (or “HAPs”), also known as toxic air pollutants or air toxics, may cause cancer or other serious health effects, such as reproductive effects or birth defects, or adverse environmental and ecological effects. HAPs are regulated in many countries. Hexane, xylene, toluene and perchloroethylene are considered HAPs.

Furthermore, many organic solvents are highly volatile and, of the total quantity released to the environment, a significant percentage eventually enters the atmosphere. As such, these solvents have been designated volatile organic compounds (or “VOCs”) and are regulated. Compounds or solvents having lower volatility have been classified as VOC-exempt in the United States (U.S.) by the Environmental Protection Agency (EPA), and/or the South Coast Air Quality Management District (SCAQMD) of California and in Canada by the National Pollutant Release Inventory (NPRI). Hexane is a VOC emitter.

### SUMMARY OF THE INVENTION

The present disclosure provides, in part, a solvent composition including para-chlorobenzotrifluoride (PCBTF), a methylated organosilicon compound, and an acetate ester.

In one aspect, the present disclosure provides a solvent composition including para-chlorobenzotrifluoride (PCBTF), in an amount of about 40% to about 60% by volume of the solvent composition; a methylated organosilicon compound in an amount of about 25% to about 60% by volume of the solvent composition; and an acetate ester in an amount of about 0% to about 15% by volume of the solvent composition.

In some embodiments, the para-chlorobenzotrifluoride (PCBTF) may include about 45% to about 60% by volume of the solvent composition; the methylated organosilicon compound may include about 30% to about 55% by volume of the solvent composition; and the acetate ester may include about 0% to about 15% by volume of the solvent composition.

In alternative embodiments, the para-chlorobenzotrifluoride (PCBTF) may include about 50% by volume of the solvent composition; the methylated organosilicon compound may include about 40% by volume of the solvent composition; and the acetate ester may include about 10% by volume of the solvent composition.

In alternative embodiments, the para-chlorobenzotrifluoride (PCBTF) may include about 45% by volume of the solvent composition; the methylated organosilicon compound may include about 40% by volume of the solvent composition; and the acetate ester may include about 15% by volume of the solvent composition.

In some embodiments, the acetate ester may be VOC-exempt. In some embodiments, the acetate ester may be methyl acetate (MA).

In some embodiments, the methylated organosilicon compound may be VOC-exempt. In some embodiments, the methylated organosilicon compound may be hexamethyldisiloxane (HMDS), octamethyltrisiloxane (OMTS), or decamethyltetrasiloxane (DMTS).

In some embodiments, the para-chlorobenzotrifluoride (PCBTF) may include about 50% by volume of the solvent composition; the methylated organosilicon compound may be hexamethyldisiloxane (HMDS), and may include about 40% by volume of the solvent composition; and the acetate ester may be methyl acetate (MA) and may include about 10% by volume of the solvent composition.

In some embodiments, the para-chlorobenzotrifluoride (PCBTF) may include about 45% by volume of the solvent composition; the methylated organosilicon compound may be hexamethyldisiloxane (HMDS), and may include about 40% by volume of the solvent composition; and the acetate ester may be methyl acetate (MA) and may include about 15% by volume of the solvent composition.

In some embodiments, the solvent composition may have a flash point of at least 15° C.

In some embodiments, the solvent composition may have an evaporation rate of at most 3.5.

In some aspects, the present disclosure provides a kit or commercial package comprising a solvent composition, as described herein, together with instructions for use.

In some embodiments, the present disclosure provides a solvent composition, as described herein, for use as a hexane substitute by volume.

In some embodiments, the present disclosure provides a solvent composition, as described herein, for use as a solvent.

In some embodiments, the present disclosure provides a solvent composition, as described herein, for use as a primary or co-solvent for paints, varnishes, coatings, inks, adhesives, laboratory wipes and/or equipment wipes.

In some embodiments, the present disclosure provides a solvent composition, as described herein, for use in the manufacturing and/or formulation of paints, varnishes, coatings, polymers, resins, inks, adhesives, hard surface cleaners, household dyes, tints, insecticides, lubricating greases and oils, automotive chemicals, markers, shoe polish, undercoats, and/or particleboard.

In some embodiments, the present disclosure provides a solvent composition, as described herein, for use in one or more of: reformulation of an aerosol; dissolution of rubbers; dissolution of polymers; and/or dissolution of resins.

In some embodiments, the present disclosure provides a solvent composition, as described herein, for use as a paint thinner, paint remover, cleaner, degreaser, and/or adhesive remover.

Other aspects and features of the present disclosure will become apparent to those ordinarily skilled in the art upon review of the following description.

### DETAILED DESCRIPTION

The present disclosure provides, in part, a solvent composition including para-chlorobenzotrifluoride (PCBTF), a methylated organosilicon compound and, optionally, an acetate ester.

para-Chlorobenzotrifluoride (PCBTF) has the formula C₇H₄CIF₃. It is an industrial solvent which is heavy (specific gravity of 1.34) with a pungent odour. PCBTF is VOC-exempt. In some embodiments, PCBTF may be present in the solvent composition in any amount between about 40% v/v to about 60% v/v, or between about 45% v/v to about 60% v/v, or between about 45% v/v to about 50% v/v, or between about 50% v/v to about 55% v/v, or between about 40% v/v to about 50% v/v, or any value in between or inclusive of the indicated ranges, for example, about 40% v/v, 41% v/v, 42% v/v, 43% v/v, 44% v/v, 45% v/v, 46% v/v, 47% v/v, 48% v/v, 49% v/v, 50% v/v, 51% v/v, 52% v/v, 53% v/v, 54% v/v, 55% v/v, 56% v/v, 57% v/v, 58% v/v, 59% v/v, 60% v/v, etc.

By “methylated organosilicon compound,” as used herein, is meant an organic compound with two or more siloxane functional groups saturated with methyl groups. The methylated organosilicon compound may be VOC-exempt.

In some embodiments, the methylated organosilicon compound may be present in the solvent composition in any amount between about 25% v/v to about 60% v/v, or between about 35% v/v to about 55% v/v, or between about 45% v/v to about 50% v/v, or between about 50% v/v to about 55% v/v, or between about 40% v/v to about 50% v/v, or any value in between or inclusive of the indicated ranges, for example, about 25% v/v, 26% v/v, 27 v/v, 28% v/v, 29% v/v, 30% v/v, 31% v/v, 32% v/v, 33% v/v, 34% v/v, 35% v/v, 36% v/v, 37% v/v, 38% v/v, 39% v/v, 40%, 41% v/v, 42% v/v, 43% v/v, 44% v/v, 45% v/v, 46% v/v, 47% v/v, 48% v/v, 49% v/v, 50% v/v, 51% v/v, 52% v/v, 53% v/v, 54% v/v, 55% v/v, 56% v/v, 57% v/v, 58% v/v, 59% v/v, 60% v/v, etc.

Hexamethyldisiloxane (HMDS) has the formula C₆H₁₈OSi₂. It is a colourless liquid and has a slight odour. HMDS is VOC exempt. In some embodiments, the methylated organosilicon compound may be HMDS, which may be present in the solvent composition in any amount between about 25% v/v to about 60% v/v, or between about 35% v/v to about 55% v/v, or between about 45% v/v to about 50% v/v, or between about 50% v/v to about 55% v/v, or between about 40% v/v to about 50% v/v, or any value in between or inclusive of the indicated ranges, for example, about 25% v/v, 26% v/v, 27 v/v, 28% v/v, 29% v/v, 30% v/v, 31% v/v, 32% v/v, 33% v/v, 34% v/v, 35% v/v, 36% v/v, 37% v/v, 38% v/v, 39% v/v, 40%, 41% v/v, 42% v/v, 43% v/v, 44% v/v, 45% v/v, 46% v/v, 47% v/v, 48% v/v, 49% v/v, 50% v/v, 51% v/v, 52% v/v, 53% v/v, 54% v/v, 55% v/v, 56% v/v, 57% v/v, 58% v/v, 59% v/v, 60% v/v, etc.

Octamethyltrisiloxane (OMTS) has the formula C₈H₂₄O₂Si₃. It is a colourless liquid and has a slight odour. OMTS is VOC exempt. In some embodiments, the methylated organosilicon compound may be OMTS, which may be present in the solvent composition in any amount between about 25% v/v to about 60% v/v, or between about 35% v/v to about 55% v/v, or between about 45% v/v to about 50% v/v, or between about 50% v/v to about 55% v/v, or between about 40% v/v to about 50% v/v, or any value in between or inclusive of the indicated ranges, for example, about 25% v/v, 26% v/v, 27 v/v, 28% v/v, 29% v/v, 30% v/v, 31% v/v, 32% v/v, 33% v/v, 34% v/v, 35% v/v, 36% v/v, 37% v/v, 38% v/v, 39% v/v, 40%, 41% v/v, 42% v/v, 43% v/v, 44% v/v, 45% v/v, 46% v/v, 47% v/v, 48% v/v, 49% v/v, 50% v/v, 51% v/vm, 52% v/v, 53% v/v, 54% v/v, 55% v/v, 56% v/v, 57% v/v, 58% v/v, 59% v/v, 60% v/v, etc.

Decamethyltetrasiloxane (DMTS) has the formula C₁₀H₃₀O₃Si₄. It is a colourless liquid and has a slight odour. DMTS is VOC-exempt. In some embodiments, the methylated organosilicon compound may be DMTS, which may be present in the solvent composition in any amount between about 25% v/v to about 60% v/v, or between about 35% v/v to about 55% v/v, or between about 45% v/v to about 50% v/v, or between about 50% v/v to about 55% v/v, or between about 40% v/v to about 50% v/v, or any value in between or inclusive of the indicated ranges, for example, about 25% v/v, 26% v/v, 27 v/v, 28% v/v, 29% v/v, 30% v/v, 31% v/v, 32% v/v, 33% v/v, 34% v/v, 35% v/v, 36% v/v, 37% v/v, 38% v/v, 39% v/v, 40%, 41% v/v, 42% v/v, 43% v/v, 44% v/v, 45% v/v, 46% v/v, 47% v/v, 48% v/v, 49% v/v, 50% v/v, 51% v/v, 52% v/v, 53% v/v, 54% v/v, 55% v/v, 56% v/v, 57% v/v, 58% v/v, 59% v/v, 60% v/v, etc.

By “acetate ester,” as used herein, is meant an acetic acid alkyl (C₁-C₄) ester having the formula CH₃CO₂R, where R is C₁-C₄ alkyl. “Alkyl” refers to a straight or branched hydrocarbon chain group consisting solely of carbon and hydrogen atoms, containing no unsaturation and including, for example, from one to four carbon atoms, such as 1, 2, 3, or 4 carbon atoms.

In some embodiments, the acetate ester (C₁-C₄) may be present in the solvent composition in any amount between about 0% v/v to about 15% v/v, or between about 0% v/v to about 5% v/v, or between about 5% v/v to about 10% v/v, or between about 10% v/v to about 15% v/v, or any value in between or inclusive of the indicated ranges, for example, about 0% v/v, 1% v/v, 2% v/v, 3% v/v, 4% v/v, 5% v/v, 6% v/v, 7% v/v, 8% v/v, 9% v/v, 10% v/v, 11% v/v, 12% v/v, 13% v/v, 14% v,v, 15v/v, etc.

Methyl acetate (MA) has the formula CH₃COOCH₃. It is a flammable liquid with a solubility of 25% in water at room temperature and is not stable in the presence of strong aqueous bases or aqueous acids. MA is VOC-exempt. In some embodiments, the acetate ester (C₁-C₄) may be MA, which may be present in the solvent composition in any amount between about 0% v/v to about 15% v/v, or between about 0% v/v to about 5% v/v, or between about 5% v/v to about 10% v/v, or between about 10% v/v to about 15% v/v, or any value in between or inclusive of the indicated ranges, for example, about 0% v/v, 1% v/v, 2% v/v, 3% v/v, 4% v/v, 5% v/v, 6% v/v, 7% v/v, 8% v/v, 9% v/v, 10% v/v, 11% v/v, 12% v/v, 13% v/v, 14% v/v, 15% v/v, etc.

tert-Butyl acetate (TBAc) has the formula C₆H₁₂O₂. It is a colorless flammable liquid with a blueberry-like smell. TBAc may be VOC-exempt under EPA regulations. In some embodiments, the acetate ester (C₁-C₄) may be TBAc, which may be present in the solvent composition in any amount between about 0% v/v to about 15% v/v, or between about 0% v/v to about 5% v/v, or between about 5% v/v to about 10% v/v, or between about 10% v/v to about 15% v/v, or any value in between or inclusive of the indicated ranges, for example, about 0% v/v, 1% v/v, 2% v/v, 3% v/v, 4% v/v, 5% v/v, 6% v/v, 7% v/v, 8% v/v, 9% v/v, 10% v/v, 11% v/v, 12%, 13v/v, 14% v/v, 15% v/v, etc.

In some embodiments, the disclosure may provide a solvent composition including PCBTF in an amount between about 45% v/v and about 60% v/v, a methylated organosilicon compound in an amount between about 30% and about 55%, and an acetate ester in an amount between about 0% v/v and about 15% v/v.

In some embodiments, the solvent composition may include PCBTF in an amount of about 50% v/v, a methylated organosilicon compound in an amount of about 40% v/v and an acetate ester in an amount of about 10% v/v.

In alternative embodiments, the solvent composition may include PCBTF in an amount of 45% v/v, a methylated organosilicon compound in an amount of about 40% v/v and an acetate ester in an amount of about 15% v/v.

In some embodiments, a solvent composition according to the present disclosure includes PCBTF in an amount of about 50% v/v (about 62.8% wt %), HMDS in an amount of about 40% v/v (about 28.5 wt %), and MA in an amount of about 10% v/v (about 8.7 wt %).

In some embodiments, a solvent composition according to the present disclosure may include PCBTF in an amount of about 45% v/v (about 39.1% wt %), HMDS in an amount of about 40% v/v (about 50.2% wt %), and MA in an amount of about 15% v/v (about 10.7% wt %).

By “about” is meant a variance (plus or minus) from a value or range of 5% or less, for example, 0.5%, 1%, 1.5%, 2.0%, 2.5%, 3.0%, 3.5%, 4.0%, 4.5%, 5.0%, etc.

It is to be understood that varying the amount of a reagent in a solvent composition will generally require a corresponding adjustment (increase or decrease), within the specified ranges, in the amount of the other reagents in a solvent composition according to the present disclosure such that the total percentages of the reagents in the solvent composition equal 100%.

In some embodiments, while not bound to any particular theory, hexamethyldisiloxane (HMDS) may be used as an ingredient that does not contribute any hydrogen bonding capability or polarity of the composition according to the present disclosure.

In some embodiments, while not bound to any particular theory, methyl acetate (MA) may be used as an ingredient in order to increase the calculated or measured evaporation rate of a solvent composition according to the present disclosure.

In some embodiments, while not bound to any particular theory, PCBTF may be used as an ingredient to increase the calculated or measured solvency of a solvent composition according to the present disclosure.

In some embodiments, a solvent composition according to the present disclosure may include reagents that are not classified as hazardous air pollutants (HAPs), as environmentally hazardous, or as ozone-depleting (VOCs). In some embodiments, a solvent composition according to the present disclosure may include reagents declared exempt by the National Pollutant Release Inventory (NPRI) of Canada.

In some embodiments, a solvent composition according to the present disclosure may include compounds or reagents that are VOC-exempt. Such compositions may be useful in reducing VOC emissions. MA, HMDS, OMTS and PCBTF are presently VOC-exempt.

A compound's maximum incremental reactivity (MIR) value is a measure of the compound's ability to generate ozone due to photochemical degradation. The lower the MIR value, the less ozone (and, accordingly, the less smog) that is generated by the compound. In some embodiments, a solvent composition according to the present disclosure may have a MIR value lower than hexane (MIR 1.24). In some embodiments, compositions according to the present disclosure may have a MIR value of no greater than 0.06. In alternative embodiments, compositions according to the present disclosure may have a MIR value of no greater than 0.08.

Compositions having a high flash point are useful due to safety reasons, for example, during transport or manufacture or for consumer use. In some embodiments, a solvent composition according to the present disclosure may have a flash point of at least about 4.0° C., for example, at least about 5.0° C., 10.0° C., 15.0° C., 20.0° C., 25.0° C., 30.0° C., 35.0° C., 40.0° C., 45.0° C., 50.0° C., 55.0° C., or more. In some embodiments, a solvent composition according to the present disclosure may have a flash point of between about 10.0° C. to about 55.0° C., or any value in between. In some embodiments, a solvent composition according to the present disclosure may have a flash point of at least 20.7° C. In some embodiments, a solvent composition according to the present disclosure may have a flash point of about 20.7° C.

In some embodiments, a solvent composition according to the present disclosure may have low toxicity as determined, for example by one or more of oral LD₅₀ on rats, biodegradability, teratogenicity, carcinogenicity and/or hepatic and renal toxicity measurements, which can be determined using standard methods. In some embodiments, a solvent composition according to the present disclosure may contain reagents classified as non-carcinogenic. In some embodiments, a solvent composition according to the present disclosure may have an oral LD₅₀ of about 5000 mg/kg or more.

Evaporation rates can be expressed relative to the evaporation of n-butyl acetate (=1), as a standard. Evaporation rates may be calculated or experimentally determined. In some embodiments, a solvent composition according to the present disclosure may have a calculated evaporation rate between 0.5 to 3.5, or any value in between or inclusive of this range, for example, about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 at ambient or room temperatures. In some embodiments, a solvent composition according to the present disclosure may have a calculated evaporation rate of at most 2.1, 2.2, 2.3, 2.4, 2.5, 2.6, 2.7, 2.8, 2.9, or 3.0, at ambient or room temperatures. In some embodiments, a solvent composition according to the present disclosure may have an experimentally determined evaporation rate between 1.0 to 3.5, or any value in between or inclusive of this range, for example, about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 at ambient or room temperatures. In some embodiments, a solvent composition according to the present disclosure may have an experimentally determined evaporation rate of at most 2.1, 2.2, 2.3, 2.4, 2.5, 2.6, 2.7, 2.8, 2.9, or 3.0, at ambient or room temperatures. Compositions having evaporation rates within these parameters may allow for wider usage in, for example, slower evaporating paints, coatings, inks, adhesives, lubricants, etc. In some embodiments, a solvent composition according to the present disclosure may have an evaporation rate of about 2.57 at ambient or room temperatures, which is classed as Medium Volatility.

In some embodiments, a solvent composition according to the present disclosure may be substantially anhydrous, for example, containing less than 0.02 wt % water. In alternative embodiments, a solvent composition according to the present disclosure may contain less than 500 ppm of water.

In some embodiments, a solvent composition according to the present disclosure may be substantially immiscible with water.

In some embodiments, a solvent composition according to the present disclosure may have a purity of, for example, at least 99.5%, for example, at least 99.6%, 99.7%, 99.8%, 99.9%, or 100%. In alternative embodiments, PCBTF may have a purity of, for example, at least 99.5%. In alternative embodiments, HMDS may have a purity of, for example, at least 99.5%. In alternative embodiments, MA may have a purity of, for example, at least 99.5%.

In some embodiments, a solvent composition according to the present disclosure may have low viscosity. In some embodiments, a solvent composition according to the present disclosure may have a viscosity of about 0.69. In some embodiments, a solvent composition according to the present disclosure may have a viscosity similar to hexane, for example 0.31.

In some embodiments, a solvent composition according to the present disclosure may have improved solvency (Kb Value) of about 46, relative to for example, hexane, which has a kauri Butanol (solvency) value of about 29. This may, in some embodiments, permit the use of less of the solvent composition, when compared to compositions containing hexane.

In some embodiments, a solvent composition according to the present disclosure may have a solvency of 6 (according to the solvency rating set forth in Table 1) or more, as described in Table 1 herein. In some embodiments, a solvent composition according to the present disclosure may have a solvency of 8 or more, as described in Table 1 herein.

In some embodiments, a solvent composition according to the present disclosure may have a specific gravity of about 1.067 g/ml.

In some embodiments, a solvent composition according to the present disclosure may have performance characteristics approximating that of hexane, as described herein or known in the art.

In some embodiments, a solvent composition according to the present disclosure may be recycled through distillation at an appropriate temperature (for example, above the initial boiling point of approximately 88° C. (190° F.)).

In some embodiments, a solvent composition according to the present disclosure may have a mild odor. In some embodiments, compositions according to the present disclosure may include reagents that do not have an unpleasant and/or strong odor.

In some embodiments, the present disclosure provides a solvent composition consisting essentially of para-chlorobenzotrifluoride (PCBTF), a methylated organosilicon compound, and an acetate ester, as described herein. By “consisting essentially of” is meant that inert and/or neutral compounds may be present in the solvent composition without affecting its physical properties, such as flash point or evaporation rate. Accordingly, compounds that may reduce the flash point of the solvent composition below 15° C., or increase the evaporation rate over 3.5, are specifically excluded. In some embodiments, halogenating agents or certain halogen-bearing compounds, including hypohalous, activated halo substituted compounds, and halogen donors (such as tertiary butyl hypochlorite, tertiary butyl hypobromite, diethylbromomalonate, a-bromoacetophenone, bromoacetic acid, cinnamyl bromide, 1,4-dibromo-2-butene, iodoacetic acid, bromodiphenylmethane, 9-bromofluorene, diethyl bromomalonate, benzoyl bromide, cinnamyl bromide, 1,4-dibromo-2-butene, bromoacetic acid, 1,4-dibromo-2,3-butanedione, diethyl dibromomalonate, N-monohaloalkylurethane, N,N-dihaloalkylurethane, N,N-dichloroethylurethane, N,N-dibromoethylurethane, N,N-dichloropropylurethane, N,N-dibromopropylurethane, N,N-dichlorodibenzylurethane, N,N-dibromobenzylurethane dibromoacetonitrile, tribromoacetaldehyde, alpha-bromoisobutyrophenone, ethyl 2-bromoisobutyrate, α,α,α,α-tetrabromo-σ-xylene, 9,10-dibromoanthracene, N-chloroparatoluenesulphonamide, N,N-dihalogenarylsulfonamides such as N,N-dichloro-p-toluenesulfonamide, N,N-dibromotoluenesulfonamide, N,N-dichlorobenzenesulfonamide, N,N-dibromobenzenesulfonamide, halomethyl ether, thiocyanogen, iodine azide, bromine azide, iodine chloride, iodine bromide, trichloroacetic acid iodide, acetic acid bromide, iodine nitrate, alkyl hypohalite, alkyl thionylchloride, aryl thionylchloride, nitrosyl chloride, nitrosyl bromide, etc. are specifically excluded. In some embodiments, cyclohexanes are specifically excluded.

In some embodiments, a solvent composition according to the present disclosure may be useful in replacing n-hexane with a molecular formula of C₆H₁₄. The compositions can be used, for example, as a solvent in various applications. Examples of contemplated applications include, without limitation: reformulation of an aerosol to meet a reactivity limit while maintaining performance properties and reducing the formation of tropospheric ozone; in dissolution of a wide variety of rubbers and polymers; in dissolution of a resin; as a co-solvent for paints, coatings, inks, adhesives; use as a paint thinner; use as a cleaner, such as electronics, contact and brake cleaners; use as a degreaser; or use as an adhesive remover.

In some embodiments, a solvent composition according to the present disclosure may be useful in the manufacturing and formulation of paints, coatings, polymers, inks, adhesives, as well as in industrial, commercial cleaning/de-greasing applications.

In some embodiments, a solvent composition as described herein may be used to formulate glues and adhesives for diverse applications in roofing, shoe-making and leather goods industries; to assist in the removal of cooking oils from seeds; as a cleaner and degreaser both in industry and in the laboratory; in the manufacturing process of textiles and fabrics; as a brake cleaner; and in the dissolution of a wide variety of block hydrogenated and non-hydrogenated polymers, water-proofing compounds, thermoplastic acrylic resins, chlorinated rubber compounds, various waxes and natural rubbers.

In some embodiments a solvent composition as described herein may be used, without limitation, in: paints, varnish, coatings; inks; adhesives; hard surface cleaners; household dyes; tints; insecticides; lubricating greases and oils; automotive chemicals; markers; shoe polish; undercoats; particleboard; surface preparation; general and heavy duty degreasing; laboratory and equipment wipe solvents; general purpose surface wipe cleaners.

In some embodiments, a solvent composition as described herein may be used, without limitation, in: dissolution and processing of acrylic polymers and the production of resins. The resins may be, without limitation, acrylic resins, alkyd resins, phenolic resins, polyaspartic urethane resins, and epoxy resins, saturated or unsaturated polyester resins, thermoplastic acrylic and bi- and tri-block hydrogenated and non-hydrogenated polymers.

In some embodiments, a solvent composition according to the present disclosure may be useful in paints and coating formulations and/or cleaning, paint and/or varnish removers, ink and/or marker formulations and/or cleaning, adhesive formulations and/or cleaning and/or removal, gelcoat formulations and/or cleaning, fiberglass manufacturing, hard surface cleaners, undercoat formulation and/or cleaning, waterproofing compounds, household dyes and/or tints, and shoe polishes, general aerospace cleaning, lubricating oils and/or greases, automotive chemicals and cleaners, etc.

In some embodiments, a solvent composition according to the present disclosure may be useful as a surface preparation and/or precision cleaner, general purpose surface wipe cleaner (for example, prior to painting), general and/or heavy duty degreaser, laboratory and equipment wipe solvent, brake and/or contact cleaner, etc.

In some embodiments, a solvent composition as described herein may be used, without limitation, in: cleaning and/or removing wax, paint, varnish and/or coatings; cleaning fiberglass; cleaning gelcoat; cleaning and/or removing inks and/or markers; cleaning and/or removing dyes; cleaning excess oils and/or grease; cleaning brakes, brake dust and/or contacts; cleaning and/or removing adhesives; paint formulations and cleaning; ink and marker formulations and cleaning; precision cleaning applications; cleaning hard surfaces; aerospace cleaning applications; and/or general cleaning and degreasing applications.

In some embodiments, such a solvent composition may be used as a hexane replacement. In some embodiments, such a solvent composition may be used, without limitation, in dissolution and processing of acrylic polymers; the production of resins (such as acrylic resins, urethane resins, alkyd resins, phenolic resins, polyaspartic urethane resins, epoxy resins, saturated or unsaturated polyester resins); thermoplastic acrylic resins, and bi- and tri-block hydrogenated and non-hydrogenated polymers and chlorinated rubber compounds or fiberglass and/or gelcoat manufacturing; or waterproofing compounds.

In some embodiments, such a solvent composition may be used, without limitation, in paints, varnish, fiberglass and gelcoat manufacturing, paint and varnish removers, coatings, inks, adhesives, hard surface cleaners, household dyes, tints, insecticides, laundry starches, lubricating greases and oils, automotive chemicals, markers, shoe polish, undercoats, waterproofing compounds, particleboard, surface preparation, general and heavy duty degreasing, laboratory and equipment wipe solvent, and as a general purpose surface wipe cleaner.

It is to be understood that a solvent composition according to the present disclosure can be used in a variety of applications in which hexane is traditionally used, and can be used to replace hexane in such applications. Accordingly, it is to be understood that the ultimate amounts of a solvent composition according to the present disclosure may vary depending on the ultimate use and final composition of the product in which the solvent composition according to the present disclosure is being used.

### EXAMPLES

Candidate compounds were selected using a number of environmental criteria, such as low flammability, safety, VOC exempt status, and sustainable sourcing.

Candidate compounds were also selected based on their physicochemical properties as, for example, determined from manufacturers' Material Safety Data Sheets, various chemical databases, such as CHEMnetBASE or Chemspider. Candidates with relatively high flash points, low toxicity and low vapor pressures, when compared with hexane, etc. were selected for further testing.

### Example 1

A solvent composition (Formulation 1) was prepared by mixing the following:
50% (v/v) or 62.8% wt % PCBTF, 99.5% purity, (CAS 98-56-6)
40% (v/v) or 28.5% wt % HMDS, 99.5% purity, (CAS 107-46-0)
10% (v/v) or 8.7% wt % MA, ≥99% purity, (CAS 79-20-9)

Formulation 1 has a MIR value of 0.062; a predicted flash point of about 20.7° C.; and a calculated evaporation rate=2.57, which is significantly lower than hexane at 8.3.

The physical/chemical Characteristics of Formulation 1, based on weighted averages (% volume) of the individual components (with the exception of initial boiling point and evaporation rate which were determined experimentally), were as follows:

| | |
|---|---|
| Upper Explosive Limit (UEL %) | 15.6 |
| Lower Explosive Limit (LEL %) | 0.95 |
| Auto Ignition Temp (° C.) | 431.5 (808.7° F.) |
| Flashpoint (° C.) | [20.7] (69.3° F.) |
| Initial Boiling Point (° C.) | 88.0 (190.4° F.) |
| Melting Point (° C.) | −49.9 (−57.8° F.) |
| Density (g/mL @ 25° C.) | 1.067 (8.90 lb/gal) |
| Viscosity (cP @ 25° C.) | 0.69 |
| Surface Tension (dynes/cm) | 21.14 |
| Specific Gravity | 1.067 |
| Solubility in H₂O (g/mL) | 0.0242 |
| Evaporation Rate (n-Butyl Acetate = 1) | 1.4 |
| Vapour Pressure (mmHg @ 20° C.) | 36.83 |
| Vapour Density (mmHg Air = 1) | 5.60 |
| Kauri Butanol (K_{b}) Value | 46.4 |
| Maximum Incremental Reactivity | 0.0622 |
| (MIR) | |
| Purity (Wt % Min) | 99.5% |
| Water Content (ppm) | <500 |
| Colour (Alpha, max) | 5 (Clear) |
| Volatility (%) | 100 |
| Hansen solubility parameters (MPA) | |
| δD (dispersion) | 13.46 |
| δP (polar) | 5.67 |
| δH (hydrogen bonding) | 3.11 |

### Example 2

A solvent composition (Formulation 2) was prepared by mixing the following:
40% (v/v) or 52.2% wt % PCBTF, 99.5% purity, (CAS 98-56-6)
40% (v/v) or 29.6% wt % HMDS, 99.5% purity, (CAS 107-46-0)
20% (v/v) or 18.2% wt % MA, ≥99% purity, (CAS 79-20-9)

Formulation 2 has a predicted MIR value of 0.058; a predicted flash point of about 15.4° C.; and a calculated evaporation rate=3.08.

### Example 3

A solvent composition (Formulation 3) was prepared by mixing the following:
45% (v/v) or 57.6% wt % PCBTF, 99.5% purity, (CAS 98-56-6)
40% (v/v) or 29.0% wt % HMDS, 99.5% purity, (CAS 107-46-0)
15% (v/v) or 13.4% wt % MA, ≥99% purity, (CAS 79-20-9)

Formulation 3 has a predicted MIR value of 0.060; a predicted flash point of about 18.1° C.; and a calculated evaporation rate=2.83.

### Example 4

A solvent composition (Formulation 4) was prepared by mixing the following:
35% (v/v) or 48.3% wt % PCBTF, 99.5% purity, (CAS 98-56-6)
60% (v/v) or 46.9% wt % HMDS, 99.5% purity, (CAS 107-46-0)
5% (v/v) or 4.8% wt % MA, ≥99% purity, (CAS 79-20-9)

Formulation 4 has a predicted MIR value of 0.042; a predicted flash point of about 14.9° C.; and a calculated evaporation rate=2.90.

### Example 5

A solvent composition (Formulation 5) was prepared by mixing the following:
35% (v/v) or 47.8% wt % PCBTF, 99.5% purity, (CAS 98-56-6)
55% (v/v) or 42.6% wt % HMDS, 99.5% purity, (CAS 107-46-0)
10% (v/v) or 9.5% wt % MA, ≥99% purity, (CAS 79-20-9)

Formulation 5 has a predicted MIR value of 0.046; a predicted flash point of about 14.4° C.; and a calculated evaporation rate=3.00.

### Example 6

A solvent composition (Formulation 6) was prepared by mixing the following:
35% (v/v) or 47.4% wt % PCBTF, 99.5% purity, (CAS 98-56-6)
50% (v/v) or 38.4% wt % HMDS, 99.5% purity, (CAS 107-46-0)
15% (v/v) or 14.1% wt % MA, ≥99% purity, (CAS 79-20-9)

Formulation 6 has a predicted MIR value of 0.049; a predicted flash point of about 13.9° C.; and a calculated evaporation rate=3.12.

### Example 7

A solvent composition (Formulation 7) was prepared by mixing the following:
40% (v/v) or 53.6% wt % PCBTF, 99.5% purity, (CAS 98-56-6)
55% (v/v) or 41.8% wt % HMDS, 99.5% purity, (CAS 107-46-0)
5% (v/v) or 4.7% wt % MA, ≥99% purity, (CAS 79-20-9)

Formulation 7 has a predicted MIR value of 0.048; a predicted flash point of about 17.0° C.; and a calculated evaporation rate=2.75.

### Example 8

A solvent composition (Formulation 8) was prepared by mixing the following:
40% (v/v) or 53.1% wt % PCBTF, 99.5% purity, (CAS 98-56-6)
50% (v/v) or 37.7% wt % HMDS, 99.5% purity, (CAS 107-46-0)
10% (v/v) or 9.2% wt % MA, ≥99% purity, (CAS 79-20-9)

Formulation 8 has a predicted MIR value of 0.051; a predicted flash point of about 16.5° C.; a calculated evaporation rate=2.86.

### Example 9

A solvent composition (Formulation 9) was prepared by mixing the following:
40% (v/v) or 52.7% wt % PCBTF, 99.5% purity, (CAS 98-56-6)
45% (v/v) or 33.6% wt % HMDS, 99.5% purity, (CAS 107-46-0)
15% (v/v) or 13.7% wt % MA, ≥99% purity, (CAS 79-20-9)

Formulation 9 has a predicted MIR value of 0.055; a predicted flash point of about 16.0° C.; and a calculated evaporation rate=2.97.

### Example 10

A solvent composition (Formulation 10) was prepared by mixing the following:
45% (v/v) or 58.6% wt % PCBTF, 99.5% purity, (CAS 98-56-6)
50% (v/v) or 36.9% wt % HMDS, 99.5% purity, (CAS 107-46-0)
5% (v/v) or 4.5% wt % MA, ≥99% purity, (CAS 79-20-9)

Formulation 10 has a predicted MIR value of 0.053; a predicted flash point of about 19.2° C.; and a calculated evaporation rate=2.61.

### Example 11

A solvent composition (Formulation 11) was prepared by mixing the following:
45% (v/v) or 58.1% wt % PCBTF, 99.5% purity, (CAS 98-56-6)
45% (v/v) or 32.9% wt % HMDS, 99.5% purity, (CAS 107-46-0)
10% (v/v) or 9.0% wt % MA, ≥99% purity, (CAS 79-20-9)

Formulation 11 has a predicted MIR value of 0.057; a predicted flash point of about 18.6° C.; and a calculated evaporation rate=2.72.

### Example 12

A solvent composition (Formulation 12) was prepared by mixing the following:
50% (v/v) or 63.3% wt % PCBTF, 99.5% purity, (CAS 98-56-6)
45% (v/v) or 32.3% wt % HMDS, 99.5% purity, (CAS 107-46-0)
5% (v/v) or 4.4% wt % MA, ≥99% purity, (CAS 79-20-9)

Formulation 12 has a predicted MIR value of 0.059; a predicted flash point of about 21.3° C.; and a calculated evaporation rate=2.46.

### Example 13

A solvent composition (Formulation 13) was prepared by mixing the following:
50% (v/v) or 62.2% wt % PCBTF, 99.5% purity, (CAS 98-56-6)
35% (v/v) or 24.7% wt % HMDS, 99.5% purity, (CAS 107-46-0)
15% (v/v) or 13.0% wt % MA, ≥99% purity, (CAS 79-20-9)

Formulation 13 has a predicted MIR value of 0.066; a predicted flash point of about 20.2° C.; and a calculated evaporation rate=2.68.

### Example 14

A solvent composition (Formulation 14) was prepared by mixing the following:
55% (v/v) or 67.8% wt % PCBTF, 99.5% purity, (CAS 98-56-6)
40% (v/v) or 28.0% wt % HMDS, 99.5% purity, (CAS 107-46-0)
5% (v/v) or 4.3% wt % MA, ≥99% purity, (CAS 79-20-9)

Formulation 14 has a predicted MIR value of 0.064; a predicted flash point of about 23.4° C.; and a calculated evaporation rate=2.32.

### Example 15

A solvent composition (Formulation 15) was prepared by mixing the following:
55% (v/v) or 67.2% wt % PCBTF, 99.5% purity, (CAS 98-56-6)
35% (v/v) or 24.3% wt % HMDS, 99.5% purity, (CAS 107-46-0)
10% (v/v) or 8.5% wt % MA, ≥99% purity, (CAS 79-20-9)

Formulation 15 has a predicted MIR value of 0.068; a predicted flash point of about 22.9° C.; and a calculated evaporation rate=2.43.

### Example 16

A solvent composition (Formulation 16) was prepared by mixing the following:
55% (v/v) or 66.7% wt % PCBTF, 99.5% purity, (CAS 98-56-6)
30% (v/v) or 20.6% wt % HMDS, 99.5% purity, (CAS 107-46-0)
15% (v/v) or 12.7% wt % MA, ≥99% purity, (CAS 79-20-9)

Formulation 16 has a MIR value of 0.071; a predicted flash point of about 22.3° C.; and a calculated evaporation rate=2.5.

### Example 17

A solvent composition (Formulation 17) was prepared by mixing the following:
60% (v/v) or 72.0% wt % PCBTF, 99.5% purity, (CAS 98-56-6)
35% (v/v) or 23.8% wt % HMDS, 99.5% purity, (CAS 107-46-0)
5% (v/v) or 4.2% wt % MA, ≥99% purity, (CAS 79-20-9)

Formulation 17 has a predicted MIR value of 0.070; a predicted flash point of about 25.5° C.; and a calculated evaporation rate=2.17.

### Example 18

A solvent composition (Formulation 18) was prepared by mixing the following:
60% (v/v) or 71.5% wt % PCBTF, 99.5% purity, (CAS 98-56-6)
30% (v/v) or 20.3% wt % HMDS, 99.5% purity, (CAS 107-46-0)
10% (v/v) or 8.3% wt % MA, ≥99% purity, (CAS 79-20-9)

Formulation 18 has a predicted MIR value of 0.073; a predicted flash point of about 25.0° C.; and a calculated evaporation rate=2.28.

### Example 19

A solvent composition (Formulation 19) was prepared by mixing the following:
60% (v/v) or 70.9% wt % PCBTF, 99.5% purity, (CAS 98-56-6)
25% (v/v) or 16.8% wt % HMDS, 99.5% purity, (CAS 107-46-0)
15% (v/v) or 12.3% wt % MA, ≥99% purity, (CAS 79-20-9)

Formulation 19 has a predicted MIR value of 0.077; a predicted flash point of about 24.5° C.; and a calculated evaporation rate=2.39.

### Example 20

A solvent composition (Formulation 20) was prepared by mixing the following:
35% (v/v) or 48.7% wt % PCBTF, 99.5% purity, (CAS 98-56-6)
65% (v/v) or 51.3% wt % HMDS, 99.5% purity, (CAS 107-46-0)

Formulation 20 has a predicted MIR value of 0.039; a predicted flash point of about 5.4° C.; and a calculated evaporation rate=2.79.

### Example 21

A solvent composition (Formulation 21) was prepared by mixing the following:
40% (v/v) or 54.0% wt % PCBTF, 99.5% purity, (CAS 98-56-6)
60% (v/v) or 46.0% wt % HMDS, 99.5% purity, (CAS 107-46-0)

Formulation 21 has a predicted MIR value of 0.044; a predicted flash point of about 17.6° C.; and a calculated evaporation rate=2.64.

### Example 22

A solvent composition (Formulation 22) was prepared by mixing the following:
45% (v/v) or 59.1% wt % PCBTF, 99.5% purity, (CAS 98-56-6)
55% (v/v) or 40.9% wt % HMDS, 99.5% purity, (CAS 107-46-0)

Formulation 22 has a predicted MIR value of 0.050; a predicted flash point of about 19.7° C.; and a calculated evaporation rate=2.50.

### Example 23

A solvent composition (Formulation 23) was prepared by mixing the following:
50% (v/v) or 63.8% wt % PCBTF, 99.5% purity, (CAS 98-56-6)
50% (v/v) or 36.2% wt % HMDS, 99.5% purity, (CAS 107-46-0)

Formulation 23 has a predicted MIR value of 0.055; a predicted flash point of about 21.8° C.; and a calculated evaporation rate=2.35.

### Example 24

A solvent composition (Formulation 24) was prepared by mixing the following:
55% (v/v) or 68.3% wt % PCBTF, 99.5% purity, (CAS 98-56-6)
45% (v/v) or 31.7% wt % HMDS, 99.5% purity, (CAS 107-46-0)

Formulation 24 has a predicted MIR value of 0.061; a predicted flash point of about 23.9° C.; and a calculated evaporation rate=2.21.

### Example 25

A solvent composition (Formulation 25) was prepared by mixing the following:
60% (v/v) or 72.6% wt % PCBTF, 99.5% purity, (CAS 98-56-6)
40% (v/v) or 27.4% wt % HMDS, 99.5% purity, (CAS 107-46-0)

Formulation 25 has a predicted MIR value of 0.066; a predicted flash point of about 26.0° C.; and a calculated evaporation rate=2.06.

### Example 26

Formulation 26: 100% PCBTF.

### Example 27

Formulation 27: 100% hexanes.

### Example 28

A solvent composition (Formulation 28) having about:
22.85% (v/v) or 19.61% (wt %) HMDS,
74.91% (v/v) or 78.43% (wt %) MA,
   has a calculated flash point of -7.35° C. and a calculated evaporation rate of 5.51.

### Example 29

A solvent composition (Formulation 29) having about:
22.85% (v/v) or 19.61% (wt %) HMDS,
74.91% (v/v) or 78.43% (wt %) MA, and
2.24% (v/v) or 1.96% (wt %) Cyclohexane,
   has a calculated flash point of −7.80° C. and a calculated evaporation rate of 5.64.

### Example 30

A solvent composition (Formulation 30) having about:
33.58% (v/v) or 29.41% (wt %) HMDS,
64.23% (v/v) or 68.63% (wt %) MA,
   has a calculated flash point of -6.22° C., and a calculated evaporation rate of 5.26.

### Example 31

A solvent composition (Formulation 31) having about:
33.58% (v/v) or 29.41% (wt %) HMDS,
64.23% (v/v) or 68.63% (wt %) MA,
2.20% (v/v) or 1.96% (wt %) Cyclohexane,
   has a calculated flash point of −6.66° C. and a calculated evaporation rate of 5.38.

### Example 32

A solvent composition (Formulation 32) having about:
83.07% (v/v) or 78.43% (wt %) MA,
14.44% (v/v) or 19.61% (wt %) PCBTF,
   has a calculated flash point of −2.10° C. and a calculated evaporation rate of 5.28.

### Example 33

A solvent composition (Formulation 33) having about:
83.07% (v/v) or 78.43% (wt %) MA,
14.44% (v/v) or 19.61% (wt %) PCBTF,
2.48% (v/v) or 1.96% (wt %) Cyclohexane,
   has a calculated flash point of −2.59° C. and a calculated evaporation rate of 5.42.

### Example 34

A solvent composition (Formulation 34) having about:
29.07% (v/v) or 25% (wt %) HMDS,
70.53% (v/v) or 74% (wt %) MA,
   has a calculated flash point of −6.88° C., and a calculated evaporation rate of 5.48.

### Example 35

A solvent composition (Formulation 35) having about:
31.34% (v/v) or 25% (wt %) HMDS,
50.35% (v/v) or 49% (wt %) MA,
17.87% (v/v) or 25% (wt %) PCBTF,
   has a calculated flash point of 2.85° C., and a calculated evaporation rate of 4.47.

Several compounds were combined in different initial blends (Table 1). Standardized performance tests on solvency of a variety of resins, including (but not limited to) Kraton resins G1652, KR1164, KR1171, and G1726, Sukorez resin SU100S, and paraffin wax. It was determined that G1652 and SU100S were the most difficult to dissolve and so the tabulated data was restricted to these results. The solvent blends were compared to 100% (v/v) or 100.0% wt % PCBTF, 99.5% purity, (CAS 98-56-6; Formulation 26), which has a predicted MIR value of 0.11, a predicted flash point of about 43.0° C., and an evaporation rate=0.9, and to 100% (v/v), to100.0% wt % Hexanes, >98.5% purity, (CAS 110-54-3; Formulation 27), which has a predicted MIR value of 1.24, a predicted flash point of about -21° C., and an evaporation rate=8.3, as well as Formulations 28-35.

The performance tests on resins were performed as follows: The addition of 0.5 mL of each formulation to an individual screw-cap vial containing a stirbar and resin (G1652: ˜20 mg, SU100S: ˜40 mg, G1726: ˜25 mg, KR1164: ˜20 mg, KR1171, ˜20 mg, paraffin wax: ˜55 mg). The vials were tightly capped and solutions were stirred on a stirplate (400 rpm) at 22° C. for 1 hour. The stirbar was removed and solvency was observed, photos of the results were taken (Formulations 1-27, G1652 and SU100S). The odor of the blends was also tested empirically. Evaporation rates were calculated based on weight averages (% by volume) of individual components.

Table 1

TABLE 1

| | | p- | | | | | | | Calculated |
|---|---|---|---|---|---|---|---|---|---|
| Formulation | Hexamethyl | Chlorobenzo | Methyl | | | G1652 | SU100S | Calculated | Evaporation |
| No. | disiloxane | trifluoride | Acetate | Cyclohexane | Hexane | solvency* | solvency* | Flash Point | rate |
| 1 | 40 | 50 | 10 | 0 | 0 | 10 | 7 | 20.74° C. | 2.57 |
| 2 | 40 | 40 | 20 | 0 | 0 | 3 | 5 | 15.44° C. | 3.08 |
| 3 | 40 | 45 | 15 | 0 | 0 | 9 | 6 | 18.09° C. | 2.83 |
| 4 | 60 | 35 | 5 | 0 | 0 | 5 | 5 | 14.91° C. | 2.90 |
| 5 | 55 | 35 | 10 | 0 | 0 | 5 | 5 | 14.38° C. | 3.01 |
| 6 | 50 | 35 | 15 | 0 | 0 | 4 | 5 | 13.85° C. | 3.12 |
| 7 | 55 | 40 | 5 | 0 | 0 | 8 | 5 | 17.03° C. | 2.75 |
| 8 | 50 | 40 | 10 | 0 | 0 | 8 | 5 | 16.5° C. | 2.86 |
| 9 | 45 | 40 | 15 | 0 | 0 | 6 | 5 | 15.97° C. | 2.97 |
| 10 | 50 | 45 | 5 | 0 | 0 | 10 | 6 | 19.15° C. | 2.61 |
| 11 | 45 | 45 | 10 | 0 | 0 | 10 | 7 | 18.62° C. | 2.72 |
| 12 | 45 | 50 | 5 | 0 | 0 | 10 | 7 | 21.27° C. | 2.46 |
| 13 | 35 | 50 | 15 | 0 | 0 | 9 | 6 | 20.21° C. | 2.68 |
| 14 | 40 | 55 | 5 | 0 | 0 | 10 | 8 | 23.39° C. | 2.32 |
| 15 | 35 | 55 | 10 | 0 | 0 | 10 | 8 | 22.86° C. | 2.43 |
| 16 | 30 | 55 | 15 | 0 | 0 | 9 | 6 | 22.33° C. | 2.54 |
| 17 | 35 | 60 | 5 | 0 | 0 | 10 | 9 | 25.51° C. | 2.17 |
| 18 | 30 | 60 | 10 | 0 | 0 | 10 | 9 | 24.98° C. | 2.28 |
| 19 | 25 | 60 | 15 | 0 | 0 | 10 | 7 | 24.45° C. | 2.39 |
| 20 | 65 | 35 | 0 | 0 | 0 | 5 | 6 | 15.44° C. | 2.79 |
| 21 | 60 | 40 | 0 | 0 | 0 | 7 | 7 | 17.56° C. | 2.64 |
| 22 | 55 | 45 | 0 | 0 | 0 | 10 | 8 | 19.68° C. | 2.50 |
| 23 | 50 | 50 | 0 | 0 | 0 | 10 | 8 | 21.8° C. | 2.35 |
| 24 | 45 | 55 | 0 | 0 | 0 | 10 | 9 | 23.92° C. | 2.21 |
| 25 | 40 | 60 | 0 | 0 | 0 | 10 | 10 | 26.04° C. | 2.06 |
| 26 | 0 | 100 | 0 | 0 | 0 | 10 | 10 | 43° C. | 0.9 |
| 27 | 0 | 0 | 0 | 0 | 100 | 10 | 10 | −26° C. | 8.3 |
| 28 | 22.85 | 0 | 74.91 | 0 | 0 | — | — | −7.35° C. | 5.51 |
| 29 | 22.85 | 0 | 7.91 | 2.24 | 0 | — | — | −7.80° C. | 5.64 |
| 30 | 33.58 | 0 | 64.23 | 0 | 0 | — | — | −6.22° C. | 5.26 |
| 31 | 33.58 | 0 | 64.23 | 2.20 | 0 | — | — | −6.66° C. | 5.38 |
| 32 | 0 | 14.44 | 83.07 | 0 | 0 | — | — | −2.10° C. | 5.28 |
| 33 | 0 | 14.44 | 83.07 | 2.48 | 0 | — | — | −2.59° C. | 5.42 |
| 34 | 29.07 | 0 | 70.53 | 0 | 0 | — | — | −6.88° C. | 5.48 |
| 35 | 31.34 | 17.87 | 50.35 | 0 | 0 | — | — | 2.85° C. | 4.47 |

*Solvency scale: 1 = completely insoluble, 10 = completely soluble; equal to/greater than 6 is soluble.

Formulations containing 35% PCBTF (Formulations 4-6, 20) were not observed to dissolve the G1652 resin. Additionally, formulations 4-6 were not observed to dissolve the SU100S resin.

Formulations containing 40% PCBTF (Formulations 2, 7-9, 21) displayed varying solubility for the G1652 resin. Formulation 2 did not dissolve the G1652 resin, and formulations 7-9, 21 showed moderate to good solubility for the G1652 resin. Formulations 2, 7-9 did not dissolve the SU100S resin, and formulation 21 showed moderate solubility of the SU100S resin.

Formulations with 45% (v/v) PCBTF and 0-10% (v/v) MA (Formulations 10, 11, 22) were observed to completely dissolve the G1652 resin. Formulation 3 gave almost complete dissolution of the G1652 resin. Formulations 3 and 10 displayed good solvency of the SU100S resin, and formulation 11 was slightly better at dissolving the SU100S resin. Formulation 22 exhibited good solvency of the SU1005 resin.

Formulations with 50% (v/v) PCBTF (Formulations 1, 12, 13, and 23) were all observed to completely dissolve the G1652 resin. Formulations 1, 12 and 13 displayed good solvency of the SU100S resin, while formulation 23 was observed to have excellent solvency of the SU100S resin.

Formulations with 55% (v/v) PCBTF (Formulations 14, 15, 16 and 24) were observed to dissolve the G1652 resin. Formulations 14 and 15 displayed excellent solvency of the SU100S resin, while Formulation 16 displayed moderate solvency. Formulation 24 displayed near-complete solvency of the SU100S resin.

Formulations with 60% (v/v) PCBTF (Formulations 17, 18, 19 and 25) were observed to completely dissolve the G1652 resin. Formulation 19 displayed good solvency of the SU100S resin, and formulations 17 and 18 displayed near-complete dissolution of the SU1005 resin. Formulation 25 dissolved the SU1005 completely.

Formulations 1 to 25 exhibited superior flash points and evaporation rates compared to Formulations 28 to 35. More specifically, Formulations 1-25 exhibited significantly higher flash points than formulations 28-35. Additionally, formulations 1-25 exhibit calculated evaporation rates that are lower than formulations 28-35.

In the preceding description, for purposes of explanation, numerous details are set forth in order to provide a thorough understanding of the examples. However, it will be apparent to one skilled in the art that these specific details are not required.

The above-described examples are intended to be exemplary only. Alterations, modifications and variations can be effected to the particular examples by those of skill in the art without departing from the scope, which is defined by the claims appended hereto.

All citations are hereby incorporated by reference.

1. A solvent composition comprising:
i) para-Chlorobenzotrifluoride (PCBTF) comprising about 40% to about 60% by volume of the solvent composition;
ii) a methylated organosilicon compound comprising about 25% to about 60% by volume of the solvent composition; and
iii) an acetate ester comprising about 0% to about 15% by volume of the solvent composition.

2. The solvent composition of claim 1 wherein:
i) the para-Chlorobenzotrifluoride (PCBTF) comprises about 45% to about 60% by volume of the solvent composition;
ii) the methylated organosilicon compound comprises about 30% to about 55% by volume of the solvent composition; and
iii) the acetate ester comprises about 0% to about 15% by volume of the solvent composition.

3. The solvent composition of claim 1 wherein:
i) the para-Chlorobenzotrifluoride (PCBTF) comprises about 50% by volume of the solvent composition;
ii) the methylated organosilicon compound comprises about 40% by volume of the solvent composition; and
iii) the acetate ester comprises about 10% by volume of the solvent composition.

4. The solvent composition of claim 1 wherein:
i) the para-Chlorobenzotrifluoride (PCBTF) comprises about 45% by volume of the solvent composition;
ii) the methylated organosilicon compound comprises about 40% by volume of the solvent composition; and
iii) the acetate ester comprises about 15% by volume of the solvent composition.

5. The solvent composition of claim 1 wherein the methylated organosilicon compound or the acetate ester is VOC-exempt.

6. The solvent composition of claim 1 wherein the acetate ester is methyl acetate (MA).

7. The solvent composition of claim 1 wherein the methylated organosilicon compound is VOC-exempt.

8. The solvent composition of claim 1 wherein the methylated organosilicon compound is hexamethyldisiloxane (HMDS), octamethyltrisiloxane (OMTS), or decamethyltetrasiloxane (DMTS).

9. The solvent composition of claim 1 wherein:
i) the para-Chlorobenzotrifluoride (PCBTF) comprises about 50% by volume of the solvent composition;
ii) the methylated organosilicon compound is hexamethyldisiloxane (HMDS), and comprises about 40% by volume of the solvent composition; and
iii) the acetate ester is methyl acetate (MA) and comprises about 10% by volume of the solvent composition.

10. The solvent composition of claim 1 wherein:
i) the para-Chlorobenzotrifluoride (PCBTF) comprises about 45% by volume of the solvent composition;
ii) the methylated organosilicon compound is hexamethyldisiloxane (HMDS), and comprises about 40% by volume of the solvent composition; and
iii) the acetate ester is methyl acetate (MA) and comprises about 15% by volume of the solvent composition.

11. The solvent composition of claim 1 wherein the solvent composition has a flash point of at least 15° C.

12. The solvent composition of claim 1 wherein the solvent composition has an evaporation rate of at most 3.5.

13. A kit or commercial package comprising the solvent composition claim 1 together with instructions for use.

14. The solvent composition of claim 1 wherein the solvent composition is for use as a hexane substitute by volume.

15. The solvent composition or kit of claim 1 for use as a solvent.

16. The solvent composition or kit of claim 1 for use as a primary or co-solvent for paints, varnishes, coatings, inks, adhesives, laboratory wipes and/or equipment wipes,

17. The solvent composition or kit of claim 1 for use in the manufacturing and/or formulation of paints, varnishes, coatings, polymers, resins, inks, adhesives, hard surface cleaners, household dyes, tints, insecticides, lubricating greases and oils, automotive chemicals, markers, shoe polish, undercoats, and/or particleboard.

18. The solvent composition or kit of claim 1 for use in one or more of: reformulation of an aerosol; dissolution of rubbers; dissolution of polymers; or dissolution of resins.

19. The solvent composition or kit claim 1 for use as a paint thinner, paint remover, cleaner, degreaser, and/or adhesive remover.
